(19) United States
(12) Patent Application Publication
(10) Pub. No.: US 2025/0025099 A1
(43) Pub. Date: Jan. 23, 2025

(54) METHOD AND DEVICE FOR MITIGATING CYCLE RELATED INJURY

(71) Applicant: SAMSUNG ELECTRONICS CO., LTD., Suwon-si (KR)

(72) Inventors: Vidushi CHAUDHARY, Noida (IN); Vijayanand KUMAR, Noida (IN)

(21) Appl. No.: 18/641,603

(22) Filed: Apr. 22, 2024

Related U.S. Application Data
(63) Continuation of application No. PCT/KR2024/004293, filed on Apr. 3, 2024.

(30) Foreign Application Priority Data
Jul. 20, 2023 (IN) 202311049106

(51) Int. Cl. A61B 5/00; A61B 5/11; A61B 5/16

(52) U.S. Cl. CPC A61B 5/486; A61B 5/112; A61B 5/165; A61B 5/7275

(57) ABSTRACT

The disclosure relates to a method for mitigating cycle related injury by a device. The method includes: measuring data including at least one of gait pattern, cognitive pattern, and one or more physiological parameters of user from one or more sensors; determining one or more cycling phase and data related to the one or more cycling phase based on the measured data; measuring expected measure and general impact measure for an injury of the user during cycling session to determine injury probability of the user based on the expected measure and the general impact measure; and recommending at least one of a phase change and at least one associated activity to the user based on the determined injury probability.

### CROSS-REFERENCE TO RELATED APPLICATIONS

This application is a continuation of International Application No. PCT/KR2024/004293 designating the United States, filed on Apr. 3, 2024, in the Korean Intellectual Property Receiving Office and claiming priority to Indian patent application No. 202311049106, filed on Jul. 20, 2023, in the Indian Patent Office, the disclosures of each of which are incorporated by reference herein in their entireties.

### BACKGROUND

### Field

The disclosure relates generally to safety and mitigating injury, and for example, to a method and a device for mitigating cycle related injury.

### Description of Related Art

Cycling is a popular form of exercise and transportation taken up by fitness enthusiasts and those looking to maintain a healthy lifestyle. Cycling offers many physical benefits, such as improved cardiovascular health, lower blood pressure, and increased muscle strength. Furthermore, cycling is not only beneficial for physical health but also provides positive effects on mental health and well-being.

In addition to its physical and mental health benefits, cycling is also an environment friendly mode of transportation.

While cycling is a generally safe activity, there are some risks associated with it, including the possibility of injury, particularly when user engages in cycling continuously without taking precautionary measures. The risks of injury can be due to a variety of factors, such as poor road conditions, inappropriate cycling behaviour by the user, or the user's inability to analyse their own physical and environmental conditions.

Therefore, it is important to provide a system or method that can mitigate cycle related injury.

The existing prior art discloses a mobile intelligent injury minimization system and method. The prior art further discloses a fitness tracker wearable around a portion of a user which comprises a heart rate sensor, a memory, and a microprocessor. The memory includes a current memory component and a latent memory component. The current memory component is configured to retain current data that is updatable as the user is exercising and the latent memory component is configured to retain latent data including non-individual data not specific to the user and personal data particular to the user. The non-individual data includes general historical user data. Further, the latent memory is updatable only when the tracker is within an internet coverage area. In an embodiment, the microprocessor includes a classifier. Further, the microprocessor including classifier is configured to determine an existence of an overtraining condition based on an output of the classifier utilizing data only in said memory irrespective of presence in an internet coverage area. Further, the microprocessor is configured to provide an alert to the user after determining that the overtraining condition exists according to the output of the classifier. However, the existing prior art is silent about recommending and monitoring a phase change and at least one associated activity to the user based on the injury percentage and determining impact of the phase change. Further, there is no mention of determining a safe cycling index based on remaining injury percentage post cycling session and recommending injury recovery measures to the user.

Further, the prior art discloses a system and method for monitoring safety and productivity of physical tasks. The method includes receiving signals from at least one wearable device, identifying portions of the signals corresponding to physical activities, excerpting the portions of the signals corresponding to the physical activities, and calculating risk metrics based on measurements extracted from the excerpted portions of the signals, the risk metric indicative of high risk lifting activities. However, the existing prior art is silent about recommending and monitoring a phase change and at least one associated activity to the user based on the injury percentage and determining impact of the phase change. Further, there is no mention of determining a safe cycling index based on remaining injury percentage post cycling session and recommending injury recovery measures to the user.

In addition, the prior art discloses an activity state analyzer to analyze activity state during cycling. In an embodiment, the activity state analyzer is configured to analyze an activity state of a cyclist during cycling. The activity state analyzer includes a sensor unit configured to acquire sensor data from a plurality of types of sensors including an acceleration sensor and a gyroscope sensor that are attached to a lower back of the cyclist, and a CPU configured to analyze the activity state of the cyclist during cycling based on a detection output from the plurality of types of sensors of the sensor unit. However, the existing prior art is silent about recommending and monitoring a phase change and at least one associated activity to the user based on the injury percentage and determining impact of the phase change. Further, there is no mention of determining a safe cycling index based on remaining injury percentage post cycling session and recommending injury recovery measures to the user.

Therefore, in light of the foregoing discussion, there exists a need to address the aforementioned drawbacks associated with the existing system and method for mitigating cycle related injury.

### SUMMARY

According to an embodiment of the disclosure, a method for mitigating cycle related injury by a device is provided. The method may comprises measuring data including at least one of gait pattern, cognitive pattern, and one or more physiological parameters of a user using one or more sensors; determining one or more cycling phases and data related to the one or more cycling phases based on the measured data; measuring expected measure and general impact measure for an injury of the user during cycling session to determine an injury probability of the user based on the expected measure and the general impact measure; and recommending at least one of a phase change and at least one activity to the user based on the determined injury probability.

The one or more cycling phases may include at least one of: a warm-up phase, an intense phase, and a break phase. The data related to the one or more cycling phase may include speed and duration of each cycling phase and may be notified to the user using standard recommendations or derived recommendations depending on deviation calculation of pre-cycling state with respect to baseline condition of the user.

The pre-cycling state may include the data of the user measured from the one or more sensors before cycling session, and the baseline condition includes standard values of the data for the user.

The expected measure may be taken based on a plurality of factors including at least one of terrain, speed, posture, and health vitals, with respect to a scenario, and may be measured by determining deviation in each factor and taking into account specified weight assigned to each factor.

The general impact measure may be used to determine base injury irrespective of precautionary measures taken by the user, and is measured by summing general impact for each factor.

The injury possibility of the user may be calculated by summing the expected measure and the general impact measure.

The recommended phase change may be selected from one of phases including a break phase, a recovery phase, and a warm-up phase.

The method may further comprise determining an impact of the phase change based on physiological parameters of the user; and modifying the recommended phase change based on the impact of the phase change.

Impact of the phase change may be determined based on the physiological parameters of the user and phase change recommendation being modified based on the determined impact.

The method may further comprise determining a safe cycling index based on remaining injury possibility during post cycling session; and providing at least one post cycling recommendation based on the safe cycling index.

The remaining injury possibility may be determined based on the gait pattern, the cognitive pattern, and the physiological pattern of the user, during the post cycling session.

According to an embodiment of the disclosure, a device for mitigating cycle related injury is provided. The device may comprise memory storing instructions; and at least one processor configured to, when executing the instructions, cause the device to perform operations. The operations may comprises measuring data including at least one of gait pattern, cognitive pattern, and one or more physiological parameters of a user using one or more sensors; determining one or more cycling phases and data related to the one or more cycling phases based on the measured data; measuring expected measure and general impact measure for an injury of the user during cycling session to determine an injury probability of the user based on the expected measure and the general impact measure; and recommending at least one of a phase change and at least one activity to the user based on the determined injury probability.

According to an embodiment of the disclosure, a non-transitory computer readable storage medium storing instructions is provided. The instructions, when executed by at least one processor of a device, cause the device to perform operations. The operations may comprises measuring data including at least one of gait pattern, cognitive pattern, and one or more physiological parameters of a user using one or more sensors; determining one or more cycling phases and data related to the one or more cycling phases based on the measured data; measuring expected measure and general impact measure for an injury of the user during cycling session to determine an injury probability of the user based on the expected measure and the general impact measure; and recommending at least one of a phase change and at least one activity to the user based on the determined injury probability. The foregoing summary is illustrative and is not intended to be in any way limiting. In addition to the illustrative aspects, embodiments, and features described earlier, further aspects, embodiments, and features will become apparent by reference to the drawings and the following detailed description.

### BRIEF DESCRIPTION OF THE DRAWINGS

The same reference numbers are used throughout the figures to reference like features and components. Further, the above and other aspects, features and advantages of certain embodiments of the present disclosure will be more apparent from the following detailed description, taken in conjunction with the accompanying drawings, in which:

FIG. 1 is a flowchart illustrating an example method for mitigating cycle related injury, according to various embodiments;

FIG. 2 is a block diagram illustrating an example configuration of a system performing method for mitigating cycle related injury, according to various embodiments;

FIG. 3A is a diagram illustrating an example scale of deviation for determining type of recommendations to notify the user, according to various embodiments;

FIG. 3B is a diagram illustrating an example of standard recommendations, according to various embodiments; and

FIG. 4 is a flowchart illustrating an example phase change process during cycling session, according to various embodiments.

### DETAILED DESCRIPTION

In the following description, numerous specific details are set forth in order to provide a thorough understanding of the present disclosure. It will be apparent, however, to one skilled in the art that these specific details are merely examples and are not intended to be limiting. Additionally, it may be noted that the systems and/or methods are shown in block diagram form. It is to be understood that various omissions and substitutions of equivalents may be made as circumstances may suggest or render expedient to cover various applications or implementations without departing from the spirit or the scope of the present disclosure. Further, it is to be understood that the phraseology and terminology employed herein are for the purpose of clarity of the description and should not be regarded as limiting.

Furthermore, in the present description, references to “one embodiment” or “an embodiment” may refer, for example, to a particular feature, structure, or characteristic described in connection with an embodiment being included in at least one embodiment of the present disclosure. The appearance of the phrase “in one embodiment” in various places in the disclosure is not necessarily referring to the same embodiment, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by various embodiments and not by others. Similarly, various requirements are described, which may be requirements for various embodiments but not for other embodiments.

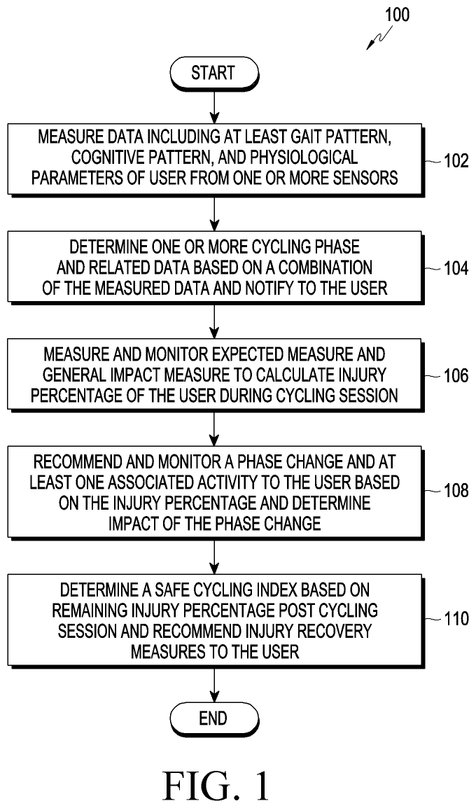

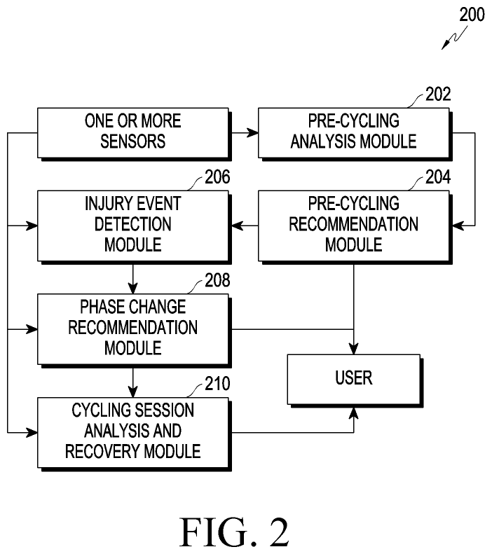

Cycling is a low-impact form of exercise that is easy on joints and can be enjoyed by people of all ages. However, cycling also entails some risks in the form of injuries, which can sometimes become significant and therefore need to be mitigated. Referring to FIG. 1, a flowchart illustrating an example method (100) for mitigating cycle related injury is disclosed according to various embodiments. The method may be explained in conjunction with the system disclosed in FIG. 2. In the flowchart, each block may represent a module, segment, or portion of code, which comprises one or more executable instructions for implementing the specified logical function(s). It should also be noted that in various alternative implementations, the functions noted in the blocks may occur out of the order noted in the drawings. For example, two blocks shown in succession in FIG. 1 may be executed substantially concurrently or the blocks may sometimes be executed in the reverse order, depending upon the functionality involved. Any process descriptions or blocks in flowcharts should be understood as representing modules, segments, or portions of code that include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure such as a state machine.

At step 102, data is measured from one or more sensors. In an embodiment, data may include at least one of gait pattern, cognitive pattern, and physiological parameters of user. The gait pattern may refer, for example, to a biological characteristic of the user that may vary based on factors such as age, body type, physical condition, and any underlying medical or neurological conditions. The gait pattern of the user may be measured to determine walking capability or state of the user such as walking before cycling, walking with cycling, etc. The cognitive pattern may refer, for example, to mental processes and behaviors of the user that relate to their ability to walk safely and effectively and may be used to determine at least, but not limited to, speed, balance, precaution level, and walking stability of the user. The physiological parameters may include measurable characteristics of the user that describe the various functions and processes taking place within the body and can be used to monitor the user's health. Examples of physiological parameters may include at least, but not limited to, hydration level, blood pressure, body temperature, breathing rate, resting heart rate, sleep duration, respiratory rate, blood oxygen saturation, and various electrophysiological readings. In an embodiment, the data including at least gait pattern, cognitive pattern, and physiological parameters of user may be determined based on the user profile. One or more cycling phase and related data is determined and notified to the user, at step 104. In an embodiment, the one or more cycling phase and related data may be determined based on a combination of the measured data. The one or more cycling phase include a warm-up phase, an intense phase, and a break phase and the data related to the one or more cycling phase includes speed and duration of each cycling phase and are notified to the user using standard recommendations or derived recommendations depending on deviation calculation of pre-cycling state with respect to baseline condition of the user. The pre-cycling state includes the data of the user measured from the one or more sensors before cycling session and baseline condition includes standard values of the data for the user.

Expected measure and general impact measure are measured and monitored to calculate injury percentage (i.e. possibility) of the user, at step 106. In an embodiment, the expected measure and general impact measure for an injury of the user are measured during cycling session and the injury percentage of the user is calculated by performing summation of the expected measure and the general impact measure The expected measure may refer, for example, to the measures taken by the user, based on a plurality of factors such as, but not limited to, terrain, speed, posture, and health vitals, with respect to a scenario, and is measured by determining deviation in each factor and taking into account predefined weightage assigned to each factor. It should be noted that the expected measure determines standard injury that may be reduced or avoided if precautions or preventive measures are taken. In an example embodiment, if there are bumps on the road, by bending down and reducing speed, the user may prevent and/or reduce the injuries. The general impact measure may determine base injury irrespective of precautionary measures taken by the user, and may be measured by summing general impact for each factor. In an example embodiment, if the user plans to do cycling for one hour, there may be a risk of injury, regardless of the precautions taken by the user.

At least one of a phase change and at least one associated activity are recommended to the user, and the recommended phase change and associated activity are monitored to determine the impact of the phase change, at step 108. In an embodiment, the recommended phase change may be selected from one of phases such as break phase, recovery phase, and warm-up phase and at least one associated activity is selected from at least one of, but not limited to, stretching, resting, drinking water, walking, and correcting posture based on the injury percentage. Further, impact of the phase change is determined depending on the physiological parameters of the user and phase change recommendation is modified based on the determined impact.

A safe cycling index may be determined and injury recovery measures are recommended to the user, at step 110. The safe cycling index may be determined based on remaining injury percentage, during post cycling session. In an embodiment, the remaining injury percentage may be determined, based on the gait pattern, the cognitive pattern, and the physiological pattern of the user, during post cycling session.

Referring to FIG. 2, a block diagram illustrating an example configuration for mitigating cycle related injury is disclosed, according to various embodiments. The system (200) comprises a pre-cycling analysis module (202) for measuring data including at least one of gait pattern, cognitive pattern, and one or more physiological parameters of the user from one or more sensors. In an embodiment, the one or more sensors include sensors of a mobile device, a wearable device, and a personal audio device to determine gait pattern, cognitive pattern, and physiological parameters. In an example embodiment, the mobile device includes at least one of, but not limited to, a mobile phone, a notebook, etc., a wearable device includes at least one of, but not limited to, a wristband, a wristwatch, an augmented reality glasses, smart glasses, or any other electronic device that can be worn as an accessory, implanted in the user's body, embedded in clothing, or tattooed on the skin, and a personal audio device includes at least one of, but not limited to, earbuds, headphones, earphones, hearing aids, head-worn audio devices, shoulder- or body-worn acoustic devices. In an embodiment, data including the gait pattern, the cognitive pattern, and the physiological parameters of user are measured for the user in pre-cycling session which is a pre-cycling state, along with their baseline condition for the user. The baseline condition includes standard values of the data for the user as disclosed in Table 1:

TABLE 1
User data measured during pre-cycling
state and their baseline condition.

| | Data | | | |
|---|---|---|---|---|
| | | | | Walking |
| Condition | Heart Rate | Sleep | Hydration | Stability |
| Pre-Cycling State | 90/120 | 7:30 hrs | 40% | Good (8) |
| Baseline | 90/110 | 8:00 hrs | 50% | Good (8) |

As shown in Table 1, the pre-cycling state includes data of the user measured from the one or more sensors during the pre-cycling session. This data includes the user's heart rate (90/120), sleep duration (7.5 hours), hydration level (40%), and walking stability (good or 8). The baseline conditions includes the standard values of the data for the user such as the heart rate (90/110), sleep (8:00 Hrs), hydration (50%), and walking stability (GOOD or 8) for the user.

The system (200) further comprises a pre-cycling recommendation module (204) for determining one or more cycling phase and related data based on a combination of the measured data. The one or more cycling phase include a warm-up phase, an intense phase, and a break phase and the data related to the one or more cycling phase includes speed and duration of each cycling phase. The pre-cycling recommendation module (204) further configured for notifying the determined one or more cycling phase and related data to the user using standard recommendations or derived recommendations depending on deviation calculation of pre-cycling state with respect to baseline condition of the user.

In case the deviation of the pre-cycling state from the baseline condition is within a comparable range, standard recommendations are provided to the user. In another case, when the deviation of the pre-cycling state from the baseline condition is outside the comparable range, derived recommendations are provided to the user. The recommendations may include duration and speed the user is required to follow during the warm-up phase and the intense phase. The warm-up phase is generally refers to the phase when the user starts cycling and the intense phase is when the user is in intense cycling state.

Table 2 discloses the data measured in the pre-cycling session and the corresponding baseline condition, along with their deviations and recommendations.

TABLE 2
Deviation calculation and respective recommendation.

| | Data | | | | |
|---|---|---|---|---|---|
| | Heart | | | Walking | Recommen- |
| Condition | Rate | Sleep | Hydration | Stability | dations |
| Pre- | 90/120 | 7:30 Hrs | 40% | 8 | |
| Cycling | | | | | |
| State | | | | | |
| Baseline | 90/110 | 8:00 Hrs | 50% | 8 | |
| Deviation | −9% | 6.2% | 10% | 0% | Standard |
| | | | | | Recommen- |
| | | | | | dations |

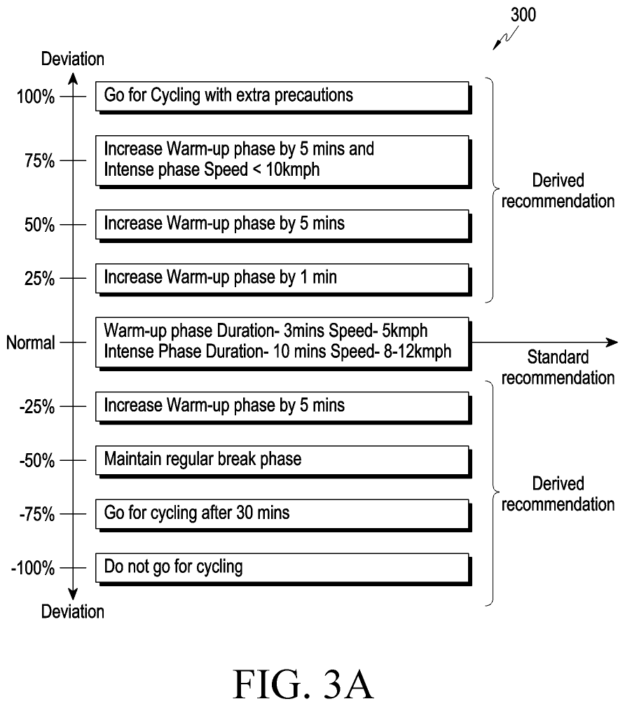

As shown in Table 2, standard recommendation is provided to the user, in case the heart rate in the pre-cycling state is deviated from the baseline condition by −9%, sleep is deviated by 6.2%, hydration is deviated by 10%, and no deviation is measured in the walking stability, considering the scale of deviation which is illustrated in FIG. 3A.

Referring to FIG. 3A, an example scale of deviation for determining type of recommendations to notify the user is illustrated according to various embodiments. As illustrated, when deviation of the pre-cycling state of the user from the baseline condition is normal or within a comparable range, the standard recommendations are provided to the user. In an example embodiment, the standard recommendation may include duration of the warm up phase as 3 mins and speed as 5 kmph and duration of the intense phase as 10 mins and speed 8-12 kmph, as illustrated in FIG. 3B. The recommendation may be provided to the user on the mobile device. In an example embodiment, the recommendation is provided to the user on the wristwatch. Further, when deviation of the pre-cycling state of the user from the baseline condition is outside the comparable range, the derived recommendation is provided to the user. The derived recommendations may vary depending upon the percentage of deviation of the pre-cycling state of the user from the baseline condition. In an example embodiment, if the deviation is-25%, a recommendation to increase the warm-up phase by 5 minutes may be given, while if the deviation is 25%, a recommendation to increase the warm-up phase by 1 minute may be given. The system (200) further comprises an injury event detection module (206). The injury event detection module (206) is configured for measuring and monitoring expected measure and general impact measure to calculate injury percentage of the user during cycling session. In an embodiment, the expected measure is taken by the user, based on a plurality of factors such as, but not limited to, terrain, speed, posture, and health vitals, with respect to a scenario. It should be noted that the injuries can occur due to any one of the factors and the expected measures can help determine standard injuries, which can be prevented or reduced by taking precautions. For example, on a bumpy road, the user may bend down and slow the speed to prevent and/or reduce the injuries.

In an embodiment, the expected measure may be measured by determining deviation in each of the factors including terrain, speed, posture, health vitals (HV) and taking into account predefined weightage assigned to each factor. Table 3 discloses the expected measure for different scenarios as shown below:

TABLE 3
Expected measure with respect to different scenario

| | Terrain | Terrain | HV | Expected |
|---|---|---|---|---|
| | w.r.t | w.r.t. | w.r.t | Measure(EM) |
| Scenario | Speed | Posture | Speed | (Weight*Deviation) |
| Weightage | 0.8 | 0.9 | 0.7 | |
| User is cycling on bumpy | | | | |
| road | | | | |
| User does not slow down | 0.9/0.2 = | — | — | 0.45 * 0.8 = 0.36 |
| on bumpy road | 0.45 | | | |
| User does not slow down | 0.9/0.2 = | 0.9/0.3 = | — | 0.45 * 0.8 + |
| on bumpy road, neither | 0.45 | 0.3 | | 0.3*0.9 = 0.63 |
| corrects the posture | | | | |
| User maintains wrong | — | 0.9/0.3 = | — | 0.3 * 0.9 = 0.27 |
| posture on bumpy road | | 0.3 | | |
| User hear rate is high and | — | — | 0.9/0.2 = | 0.45 * 0.7 = 0.31 |
| still maintains high speed | | | 0.45 | |

As shown in Table 3, the expected measure is calculated using the weightage and deviation determined for each factor during the cycling session, using the equation given below:

Expected Measure(EM)=Σweight(i)*deviation(i), for each factor

It should be noted that a low deviation indicates that no precautionary measure is being taken by the user. For instance, when there is a change in terrain, a low deviation in speed may indicate that the user has not reduced speed as a precautionary measure. Similarly, when cycling on the bumpy road, if there is a low deviation in the user's posture it may indicate that the user has not taken the precautionary measure of correcting the posture according to the terrain.

In an embodiment, the impact of the factors on different body parts of the user may be determined using the one or more mobile devices as shown in Table 4 below:

TABLE 4
Determination of impact of the each factor on different
body parts using the one or more mobile devices.

| One Or More | | | | | |
|---|---|---|---|---|---|
| Mobile | | | | Vibration | Health |
| Devices | Terrain | Speed | Posture | Intensity | Vitals |
| Wristwatch | Plain | Low | Hand | Low | All |
| | Bumpy | Medium | Elbow | Medium | (HR, RR, |
| | Hilly | High | Back | High | Stress, |
| | | | | | Sleep) |
| Wristwatch + | Plain | Low | Hand | Low | All |
| Mobile Phone | Bumpy | Medium | Elbow | Medium | |
| | Hilly | High | Back | High | |
| | | | Knee | | |
| Earbuds + | Plain | — | Knee | Low | Sleep |
| Mobile Phone | Bumpy | | Neck | Medium | |
| | Hilly | | Head | High | |
| Wristwatch + | Plain | Low | Hand | Low | All |
| Earbuds | Bumpy | Medium | Elbow | Medium | |
| | Hilly | High | Back | High | |
| | | | Neck | | |
| | | | Head | | |
| Wristwatch + | Plain | Low | Hand | Low | All |
| Earbuds + | Bumpy | Medium | Elbow | Medium | |
| Mobile Phone | Hilly | High | Back | High | |
| | | | Knee | | |
| | | | Neck | | |
| | | | Head | | |

As shown in Table 4, the wristwatch may be used to determine each of the plurality of factors including type of terrain, speed, posture of user's hand, elbow, and back as well as user's all health vitals. Further, the wristwatch along with the mobile phone may be used to determine the type of terrain, speed, posture of user's hand, elbow, back, and knee as well as user's all health vitals. Furthermore, the earbuds along with the mobile phone may be used to determine the type of terrain, posture of user's knee, neck, and head as well as user's sleep data. The wristwatch along with the earbuds may be used to determine the type of terrain, speed, posture of user's hand, elbow, back, neck, and head as well as user's all health vitals. In addition, the wristwatch along with the earbuds and mobile phone may be used to determine the type of terrain, speed, posture of user's hand, elbow, back, knee, neck, and head as well as user's all health vitals.

The general impact measure determines base injury irrespective of precautionary measures taken by the user, and is measured by summing general impact for each factor, as shown in equation below:

General Impact Measure(GI)=ΣGI(i), for each factor such as terrain, posture, speed and health vitals

It should be noted that the general impact measure determines the base injury for generic scenarios, which can be avoidable or unavoidable depending on the user and environmental condition. Table 5, discloses different scenario and respective general impact measure based on different factors.

TABLE 5
General impact measures calculation with respect to different scenarios.

| | | | | | General |
|---|---|---|---|---|---|
| | | | | Health | Impact |
| | Terrain | Speed | Posture | Vitals | Measure |
| Scenario | (GI Terrain) | (GI Speed) | (GI Posture) | (GI_HV) | (GI) |
| User is | Bumpy | — | — | — | 5% |
| cycling on | (5%) | | | | |
| bumpy road | | | | | |
| User does not | Bumpy | High | — | — | 9% |
| slow down on | (5%) | (4%) | | | |
| bumpy road | | | | | |
| User does not | Bumpy | High | Bad | — | 15% |
| slow down on | (5%) | (4%) | (6%) | | |
| bumpy road, | | | | | |
| neither corrects | | | | | |
| the posture | | | | | |
| User maintains | Bumpy | — | Bad | — | 11% |
| wrong posture | (5%) | | (6%) | | |
| on bumpy road | | | | | |
| User heart rate | — | High | — | High | 6% |
| is high and | | (4%) | | (2%) | |
| still maintains | | | | | |
| high speed | | | | | |

As shown in Table 5, cycling on the bumpy road can impact the user's body and lead to injury by up to 5%, regardless of the precautionary measures taken by the user. However, if the user does not slow down on the bumpy road, the impact may be even greater, up to 9%. This impact is due to the terrain which is impacting the user's body and leading to injury by 5%, and the speed which is impacting the user's body and leading to the injury by 4%. In order to measure the expected measures and the general impact measures, the data is accumulated and analyzed continuously during the cycling session and divided into multiple windows. In an embodiment, a sliding window approach may be used by the injury event detection module (206) for data accumulation to determine reliable data patterns for analysis for calculating the injury percentage.

It should be noted that the sliding window approach is a technique used for data accumulation in which a window of fixed or variable size moves across a sequence of data points. This window captures a subset of data points, and can be used to calculate the injury percentage.

The injury percentage of the user is calculated by performing summation of the expected measure and the general impact measure, as shown in table 6 below:

TABLE 6
Injury percentage calculation based on expected
measure and the general impact measure.

| | | General | | Injury |
|---|---|---|---|---|
| | Expected | Impact | Injury | Percent- |
| Scenario | Measure | Measure | Impact | age |
| User is cycling on bumpy | — | 0.05 | 0.05 | 5% |
| road | | | | |
| User does not slow down on | 0.36 | 0.09 | 0.45 | 45% |
| bumpy road | | | | |
| User does not slow down on | 0.63 | 0.15 | 0.78 | 78% |
| bumpy road neither corrects | | | | |
| the posture | | | | |
| User maintains wrong | 0.27 | 0.11 | 0.38 | 38% |
| posture on bumpy road | | | | |
| User hear rate is high and | 0.31 | 0.06 | 0.37 | 37% |
| still maintains high speed | | | | |

As shown in Table 6, in a scenario where the user is cycling on the bumpy road, the injury percentage is calculated to be 5% due to the general impact measure which is 0.05. In another scenario, where the user does not slow down on the bumpy road, the injury percentage is calculated to be 45% which is the percentage of summation of expected measure (0.36) and the general impact measure (0.09). Similarly, the injury percentage for other scenarios are calculated to be 78%, 38%, and 37%, depending on their respective expected measure (0.63, 0.27, and 0.31) and the general impact measure (0.15, 0.11, and 0.06).

In an embodiment, the injury event detection module (206) may be configured to determine any injuries caused by the user's fatigue level.

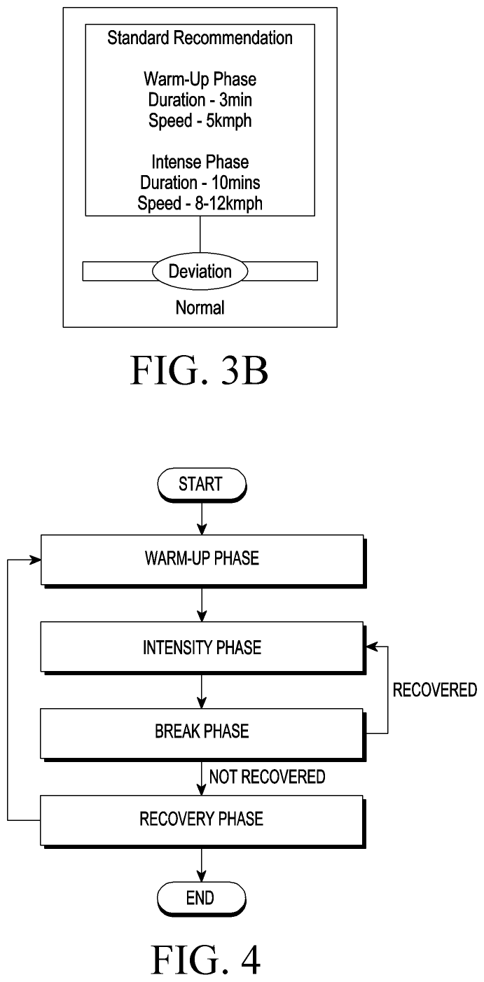

The system (200) further comprises a phase change recommendation module (208). In an embodiment, the phase change recommendation module (208) is configured for recommending a phase change and at least one associated activity to the user, based on the injury percentage during the cycling session, as illustrated in FIG. 4. The recommended phase change may be selected from one of phases such as break phase, recovery phase, and warm-up phase and at least one associated activity may include, but not limited to, stretching, resting, drinking water, walking, or correcting posture, among others.

In an embodiment, the phase change recommendation module (208) is configured for recommending the phase change, duration of the phase change, respective type of plurality of factors such as posture, terrain, speed, health vitals and environmental factors, and at least one associated activity to the user during the cycling session based on the injury percentage.

It should be noted that the break phase and the recovery phase are dependent on injury impact during the cycling session. An analysis of injury impact on the user's body, or injury severity, can help in determining the break phase and the recovery phase.

Table 7 illustrates a recommended phase change and their respective durations for different injury severity levels based on the injury percentage, as shown below:

TABLE 7
Recommended Phase change and respective duration for
each severity level based on the injury percentage.

| Severity based on injury percentage | Phase duration recommendation |
|---|---|
| Low (<20%) | Break Phase Duration <5 min |
| Mid (20-50%) | Break Phase Duration 5% of total |
| | duration |
| High (>50%) | Recovery Phase |

As shown in Table 7, severity of the injury is leveled as low, mid, and high depending upon the injury percentage, for injury percentage less than 20%, severity is low and the break phase is recommended to the user for less than 5 mins to recover. For injury percentage 20-50%, the severity is mid, and the break phase is recommended to the user for a duration equals to 5% of the total duration. For injury percentage greater than 50%, the severity is high, and the recovery phase is recommended for the user to recover from the injury.

In an example embodiment, the phase change recommendation module (208) may recommend the break phase to rest and drink water in case the user has sustained a 30% injury and body is dehydrated during the intensity phase.

Table 8 discloses different scenarios and respective phase change and at least one associated activity recommendation, as shown below:

TABLE 8
Phase change and at least one associated activity
recommendation based on the scenario.

| | Injury | Phase Change | Associated Activity |
|---|---|---|---|
| Scenario | Percentage | Recommendation | Recommendation |
| User is cycling on bumpy | 5% | Break Phase | — |
| road | | Duration <5 min | |
| User does not slow down | 45% | Break Phase | Do some stretching |
| on bumpy road | | Duration 10% of | |
| | | total duration | |
| User does not slow down | 78% | Recovery Phase | Stop cycling, |
| on bumpy road neither | | | drink some water |
| corrects the posture | | | and take rest |
| User maintains wrong | 38% | Break Phase | Do some stretching |
| posture on bumpy road | | Duration 5% of | |
| | | total duration | |
| User heart rate is high and | 37% | Break Phase | Drink water |
| still maintains high speed | | Duration 5% of | |
| | | total duration | |

As shown in Table 8, in the scenario, where the injury percentage is calculated as 5% due to cycling on a bumpy road, a break phase of less than 5 minutes is recommended to the user to recover from the injury. In another scenario, where the injury percentage is calculated as 45%, due to lack of slowing down on bumpy road while cycling on bumpy road, the break phase of duration 10% of the total duration and stretching is recommended to the user. In yet another scenario, where the injury percentage is calculated as 78%, due to lack of slowing down on bumpy road or correcting the posture while cycling on bumpy road, the recovery phase is recommended to the user which includes the recommendations such as stop cycling, drink some water and take rest for recovery from the injury. Similarly, in other scenarios where the injury percentage is calculated as 38% (due to wrong posture) and 37% (due to maintaining high speed when the heart rate is high), the break phase of duration 5% of the total duration is recommended along with stretching and drinking water respectively.

The phase change recommendation module (208) is further configured for monitoring the recommended phase change and at least one associated activity and determining impact of the phase change. In an embodiment, impact of the phase change is determined based on the physiological parameters of the user and phase change recommendation is modified based on the determined impact. In an example embodiment, if the user is recommended with the break phase and the user follows the recommendation and recovers from the injury during the break phase, the user may be recommended to go back to the intensity phase. However, if the user does not recover during the break phase, the user may be recommended to go to a recovery phase, which involves an extensive rest phase for the user's recovery. Once the user is recovered, the user may start the warm-up phase which refers to a phase for starting the cycling session.

The system (200) further comprises a cycling session analysis and recovery module (210). The cycling session analysis and recovery module (210) is configured for determining a safe cycling index based on remaining injury percentage post cycling session. The safe cycling index may be determined using the equation given below:

Safe Cycling Index=1−Remaining Injury/100

In an embodiment, the remaining injury percentage is determined, based on the gait pattern, the cognitive pattern, and the physiological pattern of the user, post cycling session. In an example embodiment, the gait pattern, the cognitive pattern, and the physiological pattern of the user are compared pre and post the cycling session to determine the remaining injury percentage.

The cycling session analysis and recovery module (210) is further configured for recommending injury recovery measures to the user post cycling session. It should be noted that the safe cycling index is calculated for each entire cycling session to determine how safely the user has cycled and also in order to facilitate effective injury recovery measures and to provide the recommendations for the next cycling session. Table 9 discloses the post cycling recommendations with respect to the safe cycling index.

TABLE 9
Post cycling recommendations with respect to the safe cycling index.

| | Remaining | Safe | | | | |
|---|---|---|---|---|---|---|
| Injury | Injury | Cycling | Recovery | | Injury | Post Cycling |
| Percentage | Percentage | Index | Time | | Impact | Recommendations |
| 5% | 0% | 1 | Immediate | | No Impact | User is ready for |
| | | | | | | next cycling session |
| 45% | 10% | 0.9 | 5 | hours | Back and | No cycling for next |
| | | | | | Elbow | 5 hours and do elbow |
| | | | | | Injury | stretching and back |
| | | | | | | massage |
| 78% | 25% | 0.75 | 1 | day | Knee, | No cycling for 1 |
| | | | | | back injury | day and do ice |
| | | | | | | massage, stretching |
| | | | | | | and take proper sleep |
| 38% | 8% | 0.92 | 5 | hour | Knee Injury | No cycling for next |
| | | | | | | 5 hours and do |
| | | | | | | stretching and take |
| | | | | | | proper sleep |

| | | | | | |
|---|---|---|---|---|---|
| 37% | 2% | 0.98 | Immediate | Dehydration | Drink water and take |
| | | | | | rest |

Table 9 shows examples of post-cycling recommendations based on the safe cycling index of the user. In an example case, where the remaining injury is 0% which signifies the safe cycling index of 1 and no impact of injury, the user is recommended for next cycling session. In another case, where the remaining injury is 10% which signifies the safe cycling index of 0.9 and impacts on back and elbow, the post cycling recommendation includes “No cycling for next 5 hours and do elbow stretching and back massage”. In yet another case, where the remaining injury is 25% which signifies the safe cycling index of 0.75 and impact on knee and back, the post cycling recommendation includes “No cycling for 1 day and do ice massage, stretching and take proper sleep” Similarly for remaining injury of 8% and 2%, the post cycling recommendation includes “No cycling for next 5 hours and do stretching and take proper sleep” and “Drink water and take rest” respectively.

It will be understood that the various modules described above may include various circuitry and/or executable program instructions for execution by a processor. The processor may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions. The modules may be referred to as at least one processor. The various modules of the system (200) may be implemented in a device. The device may further comprise a memory storing instructions. The memory may be referred to as a non-transitory computer readable storage medium. The instructions, when executed by the at least one processor of the device, cause the device to perform the operations of the modules described herein.

In an example embodiment, the present disclosure may be implemented to analyze the user's cycling experience and provide personalized recommendations prior to, during, and post cycling session for an effective and safe cycling experience. These personalized post cycling recommendations may include recommendations for stretching exercises to recover from the injury that occurred during the cycling session.

In an example embodiment, the present disclosure may be implemented to encourage the users to cycle by providing an option for social competition, where users' ranking depends on their cycling session and their safe cycling index, which motivates the users to aim for a better score and cycle more safely.

Further, the disclosure may be implemented to perform cycling trend analysis of the user to determine the most active cycling time, the most injury free cycling session, the most calories burnt cycling session, and the most unstable cycling session. Based on these analyses, the recommendations for the best time of day to cycle, the safest cycling path to take, and the best posture for cycling may be provided to the user.

Furthermore, the present disclosure may be implemented to determine cycling dynamics, which includes metrics such as seated or standing position, power phase, platform center offset, and right or left balance. It should be noted that the cycling dynamics is a collection of advanced metrics that provide a comprehensive understanding of the user's performance and how it may change based on variables such as position, bike setup, and ride duration.

In addition, the present disclosure may be implemented to provide a personalized post cycling recommendation based on an analysis of the user's cycling session, including an analysis of injury impact and the body parts which are impacted during the cycling session and analysis of the injury level. Further, the disclosure provides personalized recommendation with respect to the user's health vitals, body posture, environmental factors as well as recovery time analysis for next cycling session. While the disclosure has been illustrated and described with reference to various example embodiments, it will be understood that the various example embodiments are intended to be illustrative, not limiting. It will be further understood by those skilled in the art that the disclosed systems and methods may be modified, and all such modifications are covered by the same innovative concept, moreover, all of the details can be replaced by technically equivalent elements. The scope of protection of the disclosure includes the attached claims and equivalents. It will also be understood that any of the embodiment(s) described herein may be used in conjunction with any other embodiment(s) described herein.

What is claimed is:

1. A method for mitigating cycle related injury by a device, the method comprises:
measuring data including at least one of gait pattern, cognitive pattern, and one or more physiological parameters of a user using one or more sensors;
determining one or more cycling phases and data related to the one or more cycling phases based on the measured data;
measuring expected measure and general impact measure for an injury of the user during cycling session to determine an injury probability of the user based on the expected measure and the general impact measure; and
recommending at least one of a phase change and at least one activity to the user based on the determined injury probability.

2. The method of claim 1, wherein the one or more cycling phases include at least one of: a warm-up phase, an intense phase, and a break phase, and
the data related to the one or more cycling phase includes: speed and duration of each cycling phase and are notified to the user using standard recommendations or derived recommendations depending on deviation calculation of pre-cycling state with respect to baseline condition of the user.

3. The method of claim 2, wherein the pre-cycling state includes: the data of the user measured from the one or more sensors before cycling session, and the baseline condition includes standard values of the data for the user.

4. The method of claim 1, wherein the expected measure is taken based on a plurality of factors including at least one of terrain, speed, posture, and health vitals, with respect to a scenario, and is measured by determining deviation in each factor and taking into account specified weight assigned to each factor.

5. The method of claim 1, wherein the general impact measure is used to determine base injury irrespective of precautionary measures taken by the user, and is measured by summing general impact for each factor.

6. The method of claim 1, wherein the injury possibility of the user is calculated by summing the expected measure and the general impact measure.

7. The method of claim 1, wherein the recommended phase change is selected from one of phases including a break phase, a recovery phase, and a warm-up phase.

8. The method of claim 1, further comprising:
determining an impact of the phase change based on physiological parameters of the user; and
modifying the recommended phase change based on the impact of the phase change.

9. The method of claim 1, further comprising:
determining a safe cycling index based on remaining injury possibility during post cycling session; and
providing at least one post cycling recommendation based on the safe cycling index.

10. The method of claim 9, wherein the remaining injury possibility is determined based on at least one of the gait pattern, the cognitive pattern, and the physiological pattern of the user, during the post cycling session.

11. A device for mitigating cycle related injury, the device comprising:
memory storing instructions; and
at least one processor configured to, when executing the instructions, cause the device to perform operations comprising:
measuring data including at least one of gait pattern, cognitive pattern, and one or more physiological parameters of a user using one or more sensors;
determining one or more cycling phases and data related to the one or more cycling phases based on the measured data;
measuring expected measure and general impact measure for an injury of the user during cycling session to determine an injury probability of the user based on the expected measure and the general impact measure; and
recommending at least one of a phase change and at least one activity to the user based on the determined injury probability.

12. The device of claim 11, wherein the one or more cycling phase includes at least one of: a warm-up phase, an intense phase, and a break phase, and
the data related to the one or more cycling phase includes: speed and duration of each cycling phase and are notified to the user using standard recommendations or derived recommendations based on deviation calculation of a pre-cycling state with respect to baseline condition of the user.

13. The device of claim 12, wherein the pre-cycling state includes the data of the user measured from the one or more sensors pre-cycling, and the baseline condition includes standard values of the data for the user.

14. The device of claim 11, wherein the expected measure is taken based on a plurality of factors including at least one of terrain, speed, posture, and health vitals, with respect to a scenario, and is measured by determining deviation in each factor and taking into account specified weight assigned to each factor.

15. The device of claim 11, wherein the general impact measure is used to determine base injury irrespective of precautionary measures taken by the user, and is measured by summing general impact for each factor.

16. The device of claim 11, wherein the injury possibility of the user is calculated by summing the expected measure and the general impact measure.

17. The device of claim 11, wherein the recommended phase change includes one or more of a break phase, recovery phase, and warm-up phase.

18. The device of claim 11, wherein the operations further comprises:
determining an impact of the phase change based on physiological parameters of the user; and
modifying the recommended phase change based on the impact of the phase change.

19. The device of claim 11, wherein the operations further comprises:
determining a safe cycling index based on remaining injury possibility during post cycling session based on the gait pattern, the cognitive pattern, and the physiological pattern of the user; and
providing at least one post cycling recommendation based on the safe cycling index.

20. A non-transitory computer readable storage medium storing instructions which, when executed by at least one processor of a device, cause the device to perform operations comprising:
measuring data including at least one of gait pattern, cognitive pattern, and one or more physiological parameters of a user using one or more sensors;
determining one or more cycling phases and data related to the one or more cycling phases based on the measured data;
measuring expected measure and general impact measure for an injury of the user during cycling session to determine an injury probability of the user based on the expected measure and the general impact measure; and
recommending at least one of a phase change and at least one activity to the user based on the determined injury probability.
